(19) United States
(12) Patent Application Publication
(10) Pub. No.: US 2013/0110072 A1
(43) Pub. Date: May 2, 2013

(54) Diaper with waste bag for disposal

(71) Applicants: Evandro Carvalho, Tamarac, FL (US); Paulo Victor Carvalho, Tamarac, FL (US); Ines Maria Cockerham, Tamarac, FL (US)

(72) Inventors: Evandro Carvalho, Tamarac, FL (US); Paulo Victor Carvalho, Tamarac, FL (US); Ines Maria Cockerham, Tamarac, FL (US)

(21) Appl. No.: 13/317,816

(22) Filed: Oct. 28, 2011

(51) Int. Cl. A61F 13/84

(52) U.S. Cl. 604/385.13

(57) ABSTRACT

A diaper having a plastic bag coupled to an exterior bottom portion of the diaper is disclosed.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION BY REFERENCE OF MATERIAL SUBMITTED ON A COMPACT DISC

Not Applicable

### FIELD OF THE INVENTION

This invention relates to the field of diaper design and utility of design

### BACKGROUND OF THE INVENTION

Many people have infant, kids, elders and pets that use diapers. Unfortunately, sooner or later the new and clean diaper will become fill with human or animal waste. This waist sometimes is liquid or some times solid. The care taker of the individual must take care of changing this used diaper for a new diaper. In the process of doing it, one comes across the issue of touching it and getting the smell of it all around the environment.

One long-standing method of disposing of it is by holding it and folding it, which creates the issue of having the possibility of it unfolding and dropping waist around its surrounds.

There is the need for a disposal method which allows care takers to change the diaper and safely dispose of it without allowing waist to drop and contaminate the air and its surroundings.

### SUMMARY OF THE INVENTION

Briefly, The invention satisfies these needs by having each individual diaper be attached with a waste bag at some location of the diaper, allowing care taker to change the diaper and take it to a disposal area, also if the care taker can not dispose of it immediately he/she can carry it safely to a garbage and it also contains the smells. The diaper has a waste bag attached to it self, allowing the care taker to take the diaper of user and then pull the bag to open and push it on reverse towards the diaper, then the care taker can tie the waste bag and dispose of it.

### BRIEF DESCRIPTION OF THE DRAWINGS

The subject matter, which is regarded as the invention, is particularly pointed out and distinctly claimed in the claims at the conclusion of the specification. The foregoing and other features and also advantages of the invention will be apparent from the following detailed description taken in conjunction with the accompanying drawing. Additionally, the left-most digit of a reference number identifies the drawing in which the reference number first appears.

FIG. 1a is perspective front view of a diaper with the invention attached to its bottom.

FIG. 1b is perspective front view of a diaper with the invention attached to its bottom and with the invention been pull out, opening the bag.

FIG. 1c is perspective front view of a diaper with the invention attached to its bottom and with the invention been used to pull in the diaper inside the waste bag and the user closing the waist bag.

FIG. 2a is perspective back view of a diaper with the invention attached to its bottom.

FIG. 2b is perspective back view of a diaper with the invention attached to its bottom and with the invention been pull out, opening the bag.

FIG. 2c is perspective back view of a diaper with the invention attached to its bottom and with the invention been used to pull in the diaper inside the waste bag and the user closing the waist bag.

FIG. 3a is perspective right side view of a diaper with the invention attached to its bottom.

FIG. 3b is perspective right side view of a diaper with the invention attached to its bottom and with the invention been pull out, opening the bag.

FIG. 3c is perspective right side view of a diaper with the invention attached to its bottom and with the invention been used to pull in the diaper inside the waste bag and the user closing the waist bag.

FIG. 4a is perspective left side view of a diaper with the invention attached to its bottom.

FIG. 4b is perspective left side view of a diaper with the invention attached to its bottom and with the invention been pull out, opening the bag.

FIG. 4c is perspective left side view of a diaper with the invention attached to its bottom and with the invention been used to pull in the diaper inside the waste bag and the user closing the waist bag.

FIG. 5 is a perspective top view of a diaper, which shows that the invention makes no visible change to the inside of a diaper.

FIG. 6 is a perspective bottom view of a diaper with the invention attached to its outside bottom.

FIG. 7 is a flat angle view of the outside of a diaper with the invention attached to its center , which is the bottom of the diaper.

FIG. 8a is a top plan view of a diaper with the invention attached to its center, which is the bottom of the diaper.

FIG. 8b is a bottom plan view of a diaper with the invention attached to its center, which is the bottom of the diaper.

FIG. 8c is a left side plan view of a diaper with the invention attached to its center, which is the bottom of the diaper.

FIG. 8d is a right side plan view of a diaper with the invention attached to its center, which is the bottom of the diaper.

FIG. 9A is a perspective top view of a diaper, which shows that the invention makes no visible change to the inside of a diaper.

FIG. 9B is a perspective front view of a diaper, which shows that the invention attached to its center, which is the bottom of the diaper.

FIG. 9C is a perspective back view of a diaper, which shows that the invention attached to its center, which is the bottom of the diaper.

FIG. 9D is a perspective side view of a diaper, which shows that the invention attached to its center, which is the bottom of the diaper.

FIG. 9E is a perspective flat view of a diaper, which shows that the invention attached to its center, which is the bottom of the diaper.

FIG. 9F is a perspective front plan view of a diaper, which shows that the invention attached to its center, which is the bottom of the diaper.

### DESCRIPTION

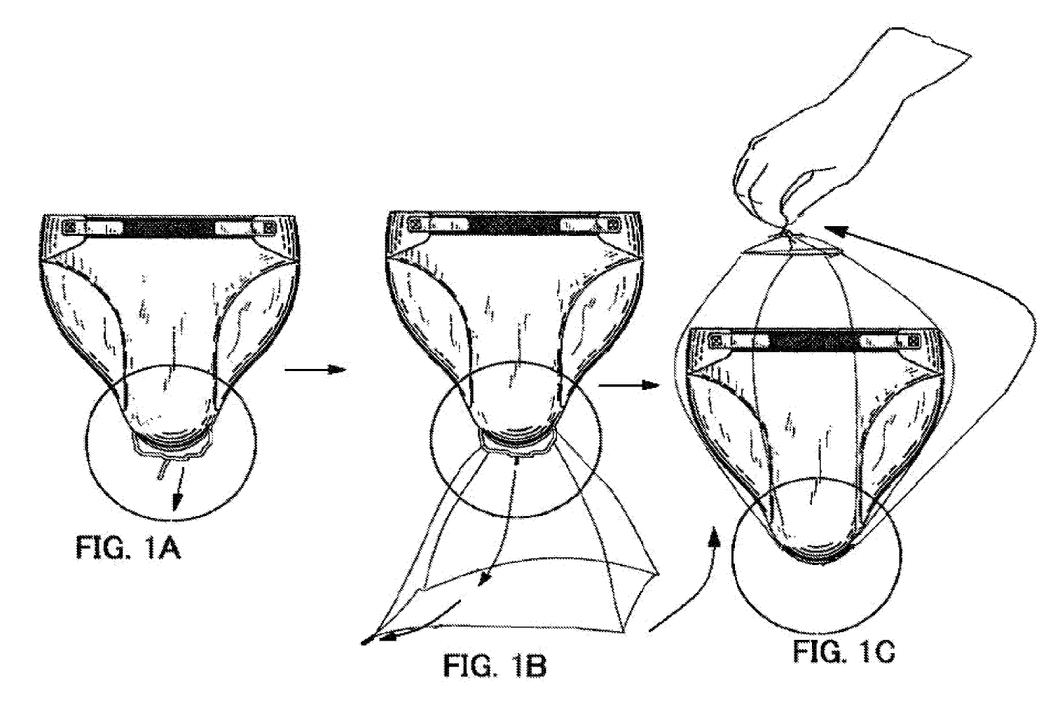

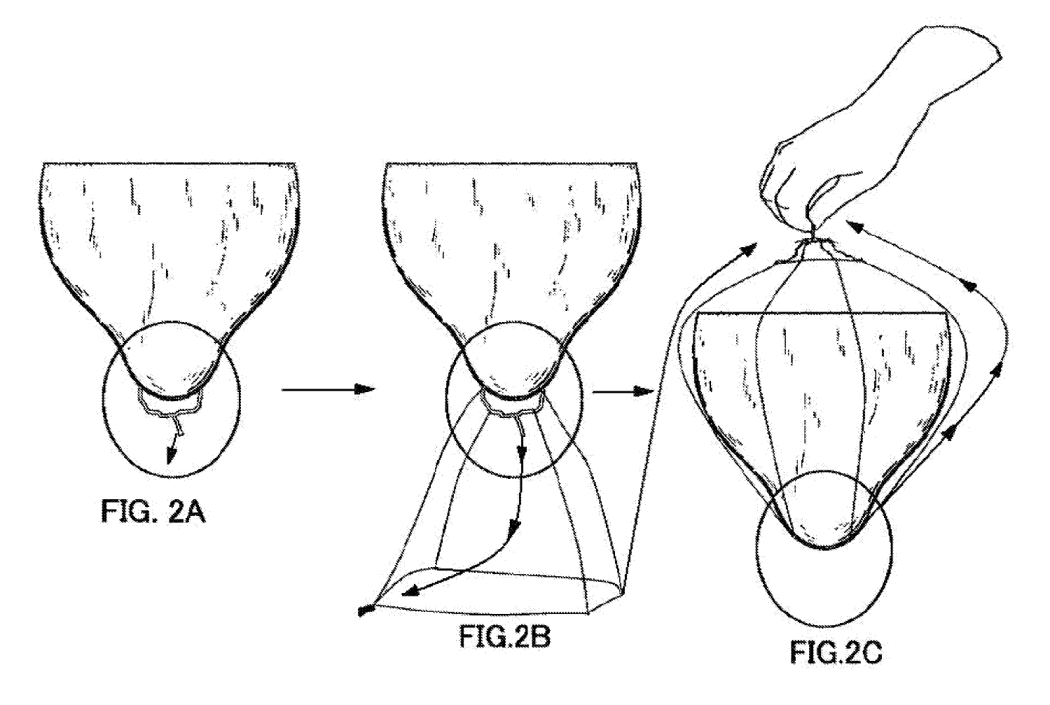

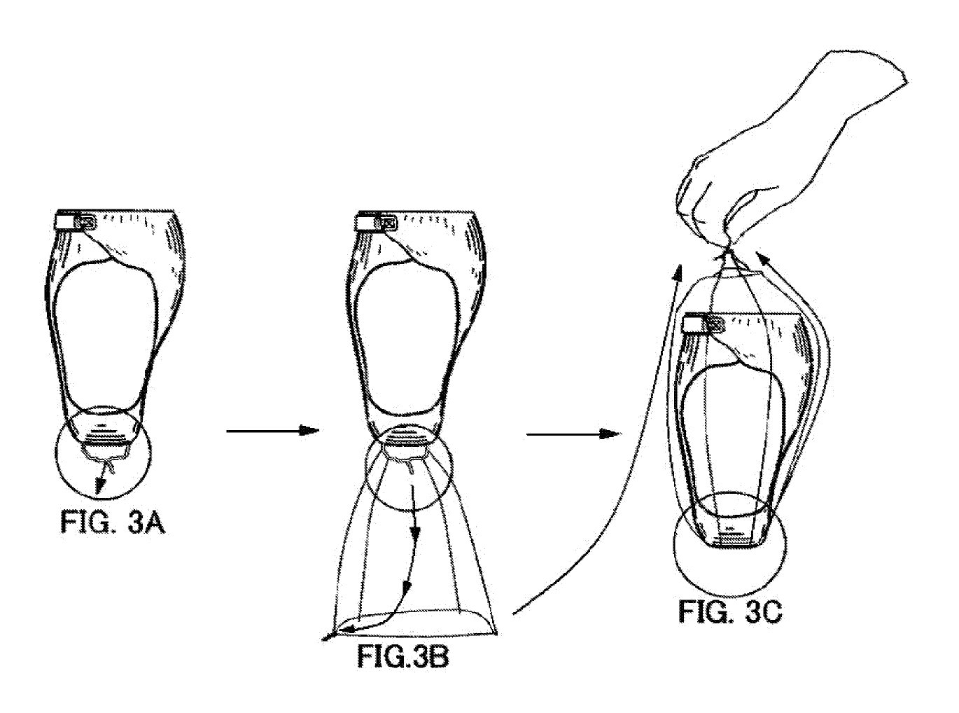

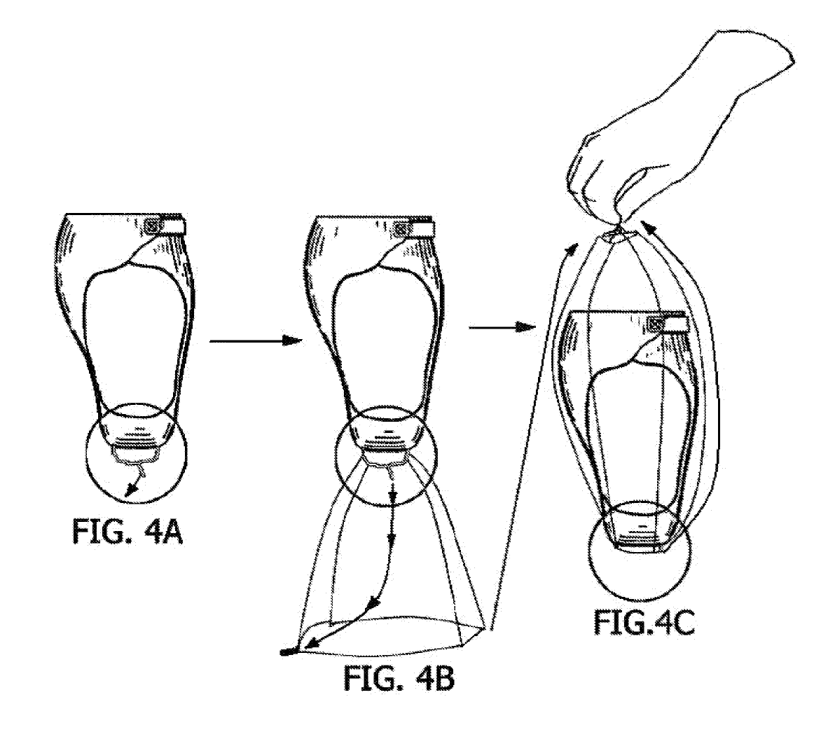

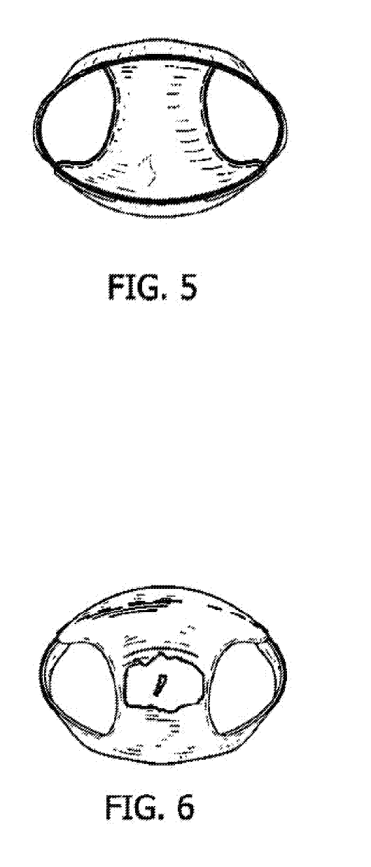

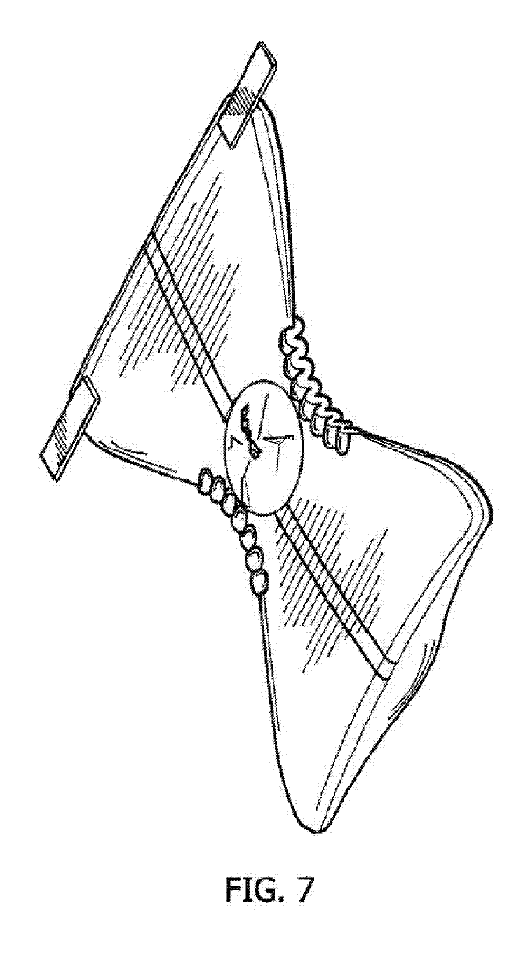

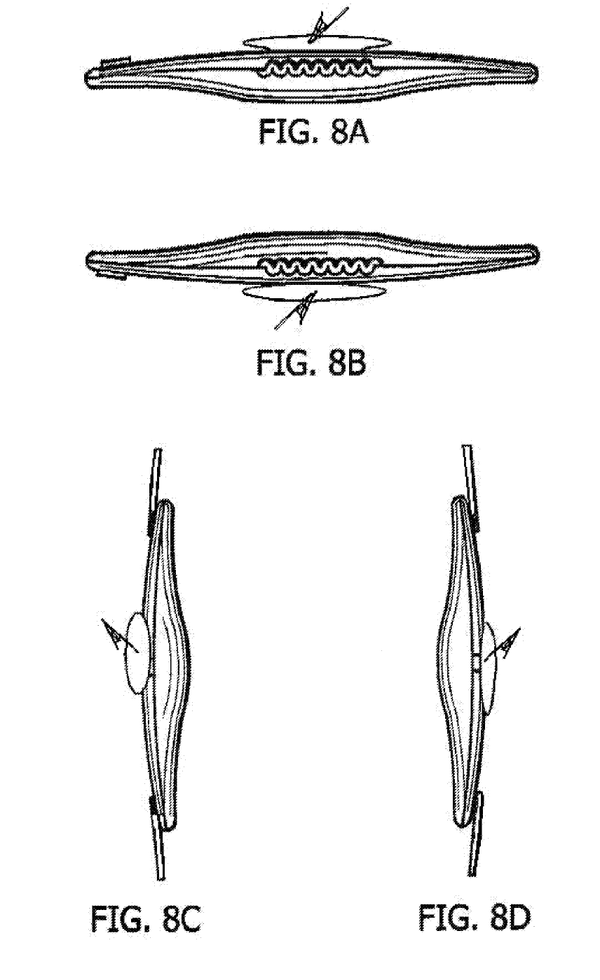

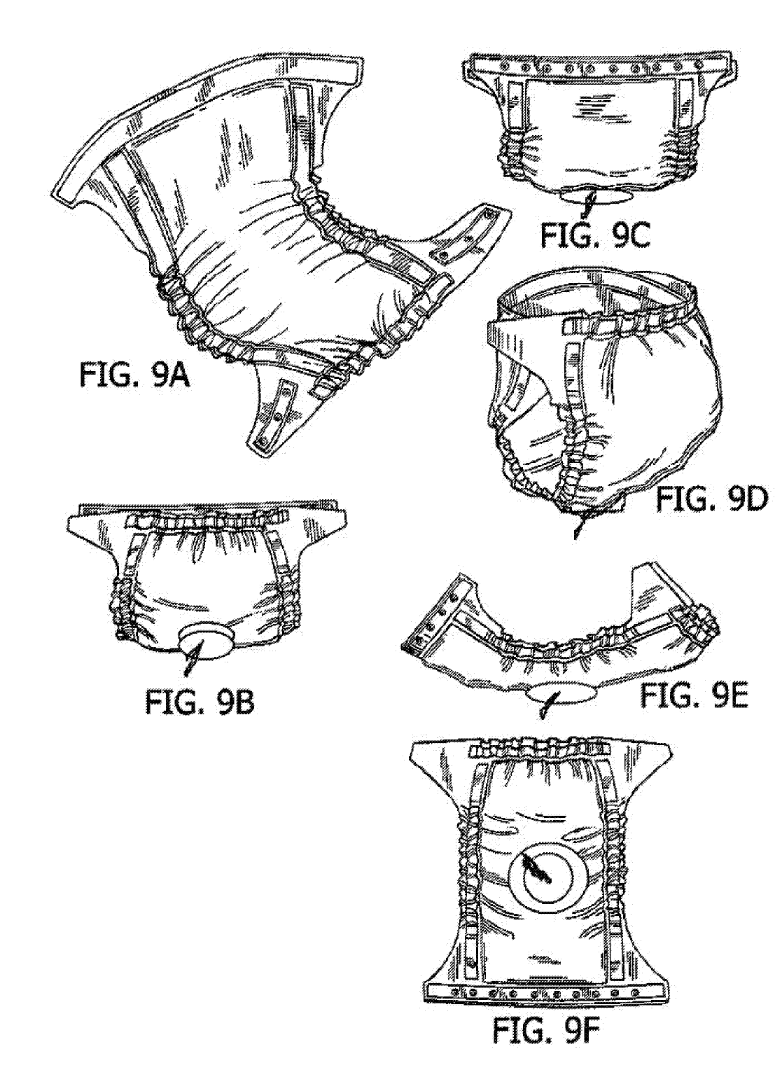

The preferred embodiment of the diaper with waste bag is shown in FIG. 1a, FIG. 1b, FIG. 1c, FIG. 2a, FIG. 2b, FIG. 2c, FIG. 3a, FIG. 3b, FIG. 3c, FIG. 4a, FIG. 4b, FIG. 4c. The invention is comprised of a waist bag attached (FIG. 1A) together to any diaper. The care taker will release the diaper from the user, then the care taker will pull (FIG. 1A) the attached waste bag from its attached location and use it to pull the diaper inside the bag (FIG. 1B) and (FIG. 1C) and then tie it up and dispose of it.

1. (canceled)

2. A diaper having a plastic bag coupled to an exterior bottom portion of the diaper.

3. The diaper of claim 2, wherein the diaper is located within a pocket coupled to an exterior bottom portion of the diaper.

4. The diaper of claim 3, wherein the pocket includes an orifice through which the diaper is pulled for use.

5. A diaper comprising:
a pocket coupled to an exterior bottom portion of the diaper;
a plastic bag disposed within the pocket; and
an orifice in the pocket through which the diaper is pulled for use.
